(19) United States
(12) Patent Application Publication
(10) Pub. No.: US 2014/0364671 A1
(43) Pub. Date: Dec. 11, 2014

(54) CATALYST MOISTURE SENSITIVTY MANAGEMENT

(71) Applicant: UOP LLC, Des Plaines, IL (US)

(72) Inventors: Wolfgang A. Spieker, Glenview, IL (US); Wei Pan, Hoffman Estates, IL (US)

(21) Appl. No.: 13/915,064

(22) Filed: Jun. 11, 2013

(51) Int. Cl. B01J 21/20; C07C 5/333

(52) U.S. Cl. CPC B01J 21/20; C07C 5/3335
USPC 585/654; 423/75

(57) ABSTRACT

A method of regenerating spent dehydrogenation catalyst is described. The spent catalyst is heated to a regeneration temperature sufficient to remove coke and adsorbed water, and exposed to a purge gas at a purge temperature sufficient to remove water vapor surrounding the regenerated catalyst, the purged catalyst being maintained at the purge temperature. A method of dehydrogenating a hydrocarbon stream using the method of regenerating spent zirconia catalyst is also described.

### BACKGROUND OF THE INVENTION

Ethylene and propylene are light olefin hydrocarbons with two or three carbon atoms per molecule, respectively, and are important chemicals for use in the production of other useful materials, such as polyethylene and polypropylene. Polyethylene and polypropylene are two of the most common plastics found in use today and have a wide variety of uses for both as a material fabrication and as a material for packaging. Other uses for ethylene and propylene include the production of vinyl chloride, ethylene oxide, ethylbenzene and alcohol. Steam cracking or pyrolysis of hydrocarbons currently produces essentially all of the ethylene and propylene. Hydrocarbons used as feedstock for light olefin production include natural gas, petroleum liquids, and carbonaceous materials including coal, recycled plastics or any organic material.

The fluidized catalyst cracking of hydrocarbons is an important process for the production of lighter hydrocarbon components, and as such, it is an important process for the production of propylene. The fluidized catalytic cracking process continuously circulates a fluidized catalyst between a reactor and a regenerator.

Another route for the production of propylene is the dehydrogenation of propane through catalytic dehydrogenation. The dehydrogenation catalysts generally comprise noble metal catalysts on acidic supports, such as alumina, or silica alumina, or zeolitic materials. However, the reaction is strongly endothermic, and requires a high temperature for the reaction to proceed at a satisfactory rate. The process also leads to coking of the catalyst, which deactivates the catalyst. The catalyst therefore needs to be regenerated on a regular basis after relatively short periods of operation, or residence, in the dehydrogenation reactor.

The production of propylene through dehydrogenation is an endothermic process and requires a substantial amount of additional heating to allow the process to proceed. The use of fuel fired charge heaters provides additional heat to the dehydrogenation reactors, but also results in problems such as fouling or coking of the charge heater tubes that can reduce the on-stream availability of the plant and increase the maintenance costs. Also, there is a loss of propylene yield that occurs due to non-selective cracking due to the thermal residence time in the heater and heater transfer line to the reactor.

Therefore, there is a need for improved methods of dehydrogenating hydrocarbons and methods of regenerating spent catalyst.

### SUMMARY OF THE INVENTION

One aspect of the invention is a method of regenerating spent dehydrogention catalyst. In one embodiment, the method includes heating the spent dehydrogenation catalyst in a regeneration zone to a regeneration temperature sufficient to remove coke and adsorbed water from the spent dehydrogenation catalyst to regenerate the spent dehydrogenation catalyst. The regenerated catalyst is separated from flue gas while maintaining the regenerated catalyst at the regeneration temperature. The regenerated catalyst is exposed to a dry purge gas at a purge temperature sufficient to remove water vapor surrounding the regenerated catalyst, the purged catalyst being maintained at the purge temperature.

Another aspect of the invention is a method of dehydrogenating a hydrocarbon stream. In one embodiment, the method includes contacting the hydrocarbon stream with a zirconia catalyst in a dehydrogenation reaction zone under dehydrogenation conditions forming a reaction product comprising olefins and spent zirconia catalyst. The reaction product and spent zirconia catalyst are removed from the dehydrogenation reaction zone. The spent zirconia catalyst is heated in a regeneration zone to a regeneration temperature sufficient to remove coke and adsorbed water from the spent zirconia catalyst to regenerate the spent zirconia catalyst. The regenerated catalyst is separated from flue gas while maintaining the regenerated catalyst at the regeneration temperature. The regenerated catalyst is exposed to a dry purge gas at a purge temperature sufficient to remove water vapor surrounding the regenerated catalyst, the purged catalyst being maintained at the purge temperature. The purged catalyst is introduced into the dehydrogenation reaction zone at the purge temperature.

### BRIEF DESCRIPTION OF THE DRAWINGS

FIG. 1 is an illustration of one embodiment of a dehydrogenation process.

FIG. 2 is an illustration of one embodiment of a dehydrogenation reaction zone and a regeneration zone.

### DETAILED DESCRIPTION OF THE INVENTION

Currently, the production of light olefins is primarily from the normal sources of light olefins that are produced through the cracking process of heavier hydrocarbons, such as naphtha or vacuum gas oil (VGO), which are produced under high severity FCC process. Light olefins are subsequently separated out from the product stream. There is a growing gap between the production of light olefins and the demand for these polymer building blocks. The demand is being met through dedicated processes that use light paraffinic feedstocks and directly convert the paraffins to olefins through dehydrogenation. Examples of suitable feedstocks include propane or an LPG feed, which can be directly dehydrogenated. This overcomes the drawbacks of other methods of light olefin production, such as methanol to olefins, and the cracking of heavier hydrocarbons.

The challenges in dehydrogenation technology include the reaction conditions, such as temperature and pressure, that favorably shift the dehydrogenation reaction equilibrium towards olefins, and the large amount of heat required to drive the reaction while minimizing undesirable side reactions, such as non-selective thermal conversion. The dehydrogenation process is endothermic and, in one current process, utilizes a plurality of reactors with inter-stage heating between the reactors. The reheating of the effluent from one reactor before passing to a subsequent reactor allows for continuous processing. The catalyst develops a coke buildup during the dehydrogenation process and must be regenerated. A continuous process includes the ability to continuously regenerate the catalyst.

Suitable processes and apparatus are described in U.S. application Ser. No. 13/681,914, filed Nov. 20, 2012, entitled Counter-current Fluidized Bed Reactor for the

Dehydrogenation of Olefins, U.S. application Ser. No. 13/681,945, filed Nov. 20, 2012, entitled Counter-Current Fluidized Bed Reactor for the Dehydrogenation of Olefins, and U.S. application Ser. No. 12/926,969, filed Nov. 1, 2010, entitled Propane Dehydrogenation Process Utilizing Fluidized Catalyst System, each of which is incorporated herein.

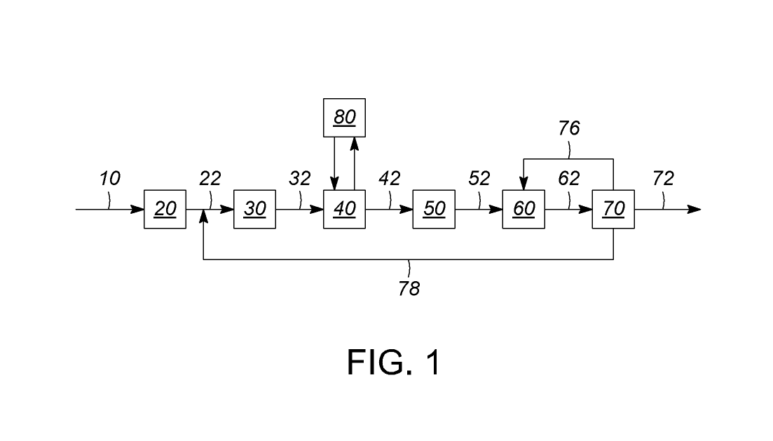

One example of a process for the production of light olefins is shown in FIG. 1. The process includes passing a hydrocarbon gas 10 that is rich in light paraffins through a dryer 20 to create a dry hydrocarbon stream 22. The dried stream 22 is passed to a heat exchanger 30 to cool the reactor products and preheat the feed. The preheated feed 32 is passed to a dehydrogenation reactor 40 to generate an intermediate stream rich in light olefins 42.

The dehydrogenation reactor 40 circulates catalyst through a catalyst inlet to the reactor 40 from a regenerator 80, and returns spent catalyst through a reactor catalyst outlet to the regenerator 80. The catalyst is heated in a combustion zone of the regenerator reactor 80 and carbon deposits on the catalyst are burned off with an oxidizing gas and supplemental fuel, to create a stream comprising catalyst and flue gas. The catalyst and flue gas are separated, and the regenerated catalyst is passed to a catalyst stripper to remove residual oxygen adsorbed onto the catalyst, and then returned to the dehydrogenation reactor 40, and the flue gas is directed to the atmosphere after catalyst and heat recovery. The stripping gas is a dry inert gas, without any significant amount of oxygen in the gas.

The intermediate stream 42 is compressed, dried and cooled in a treatment section 50 before passing the treated stream 52 to an optional selective hydrogenation reaction zone 60. In one embodiment, the treatment section consists of driers for the intermediate stream 42. The selective hydrogenation zone 60 converts dienes and acetylenes to olefins to create a hydrogenated stream 62. The hydrogenated stream 62 is passed to a separation unit 70 wherein the light olefins are separated and recovered as product stream 72.

The separation unit 70 can include a deethanizer to separate C₂ and lighter gases from the C₃ hydrocarbon stream. The deethanizer bottoms is passed to a propylene-propane splitter. The C₂ and lighter gases can also be passed to a pressure swing adsorber, to recover the hydrogen. A portion of the hydrogen 76 can be passed to the selective hydrogenation reactor 60 for use in selective hydrogenation of dienes and acetylenes.

The process can further include passing the propane rich bottoms stream 78 from the propylene-propane splitter to mix with the dry hydrocarbon feed stream 22. C₄ and heavier hydrocarbons do not need to be separated from the C₃ stream, as they will be recycled back to the dehydrogenation unit. However, depending on the feedstock composition, in the presence of relatively large amounts of C₄ and heavier hydrocarbons, the process stream can be passed through a depropanizer, with the heavier hydrocarbons passed to other process units, or recycled to the dehydrogenation reactor. A depropanizer, when added, will be located in the separation unit 70. The depropanizer is used to separate the C₄ and heavier hydrocarbons from the propane stream before passing the propane stream to the propylene/propane splitter

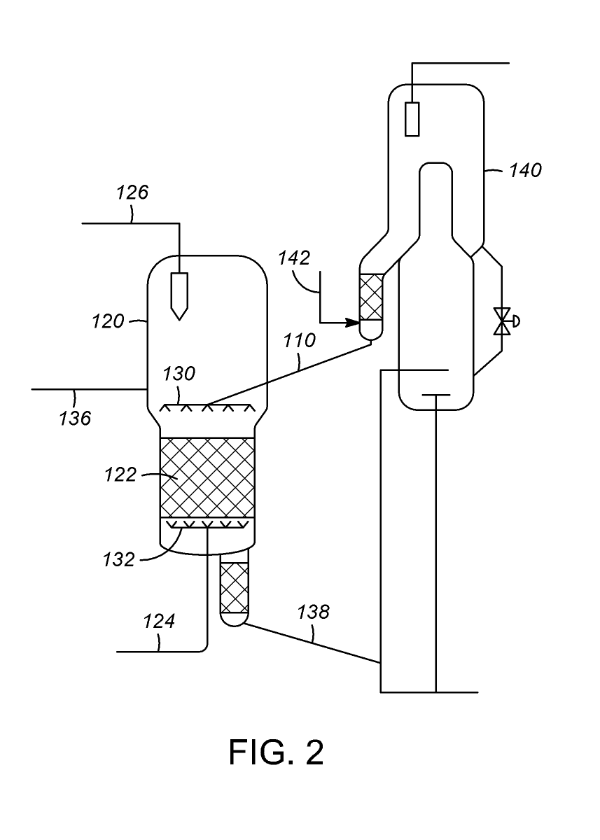

FIG. 2 illustrates one embodiment of a dehydrogenation reactor and regenerator. A regenerated catalyst stream 110 is sent to a dehydrogenation reactor 120. The catalyst flows downward through a catalyst bed 122 in the reactor 120. A paraffinic feedstream 124 is passed to the reactor 120 and flows in an upward direction through the catalyst bed 122, thereby contacting the feedstream and catalyst at dehydrogenation reaction conditions, to generate a product stream 126 comprising olefins, as well as unconverted paraffins, hydrogen, and cracking products, such as methane, ethane, and the like. Spent catalyst is collected at the bottom of the reactor 120 and transferred through a catalyst transfer line 138 and passed to a regenerator 140. The regenerator 140 regenerates the catalyst and returns the regenerated catalyst stream 110 to the reactor 120. The regenerated catalyst can undergo a stripping process with an inert gas 142 to remove residual combustion products from the regenerator. The inert gas can also be heated to maintain the catalyst temperature and to facilitate desorption of adsorbed combustion products.

The dehydrogenation process includes a reaction temperature between 400° C. and 800° C., with a temperature gradient along the axial direction of the reactor. The reactor is at its highest temperature at the top with the inlet of the regenerated catalyst and cools as the catalyst proceeds through the reactor.

The process is counter current, so the paraffinic feed, or process stream, is introduced at the bottom, or where the temperature is at its lowest. The paraffinic feed is introduced at a temperature in the range of at least about 400° C. to no greater than about 600° C., or between 450° C. and 550° C., or between 470° C. and 520° C. Partial conversion is achieved at these relatively low temperature.

The feed stream is passed to the reactor at a temperature of at least 50° C. below the catalyst inlet temperature. To maintain the temperature difference between the catalyst and the process stream, it is preferred that the paraffinic feed stream inlet temperature is between 100° C. and 250° C. below the catalyst inlet temperature, with a more preferred range of inlet temperature differences between 150° C. and 200° C. The control of the temperature differences is partially dependent upon the catalyst flow rates through the reactor and the process stream flow rates through the reactor.

This significantly reduces the amount of cracking of the hydrocarbons in the process stream, by achieving some conversion at low temperatures. The equilibrium shifts as the reaction proceeds, and, in order to continue to drive the reaction, the temperature needs to be increased to shift the equilibrium in a favorable direction. By passing the process stream counter current to the catalyst, the equilibrium adjusts favorably as the reaction proceeds, and the process stream is exposed to an increasing temperature as it passes through the reactor. The larger catalyst bed provides the heat for the reaction with the catalyst cooling as it moves downward, and allowing for initial conversion in the feedstream at a lower temperature.

The overall hot residence time is significantly reduced, and the process stream is exposed to high temperature only in the presence of catalyst for the short period of time as the process stream exits the catalyst bed. The highest temperature exposure of the process stream is also for the short contact time at the end of the process stream's residence in the reactor. The reaction conditions typically include a reactor outlet pressure between 20 kPa (absolute) and 400 kPa (absolute), or 105 kPa (absolute) to 300 kPa (absolute), or 110 kPa (absolute) to 250 kPa (absolute), or between 120 kPa (absolute) to 200 kPa (absolute).

The reactor 120 can be a larger diameter reactor, up to 11 meters, with a preferred range from 6 to 10 meters. This allows for a higher gas flow rate, while having a lower catalyst flux within the reactor. Gas flow rates within the reactor are preferred to be from 0.4 m/s to 1 m/s, with catalyst flux rates around 100,000 kg/m²/hr. The catalyst is distributed over the reactor bed with a catalyst distributor 30, and the paraffin feedstream is distributed across the bottom of the catalyst bed through a gas distributor 132.

The process further includes cooling the product stream 126. The product stream can be passed through a combined feed heat exchanger to preheat the paraffinic feed stream, and cool the product stream. The process can include passing a portion of the cooled product stream to the upper region of the reactor 120 through a quench line 136. The cooled portion of the product stream quenches the process stream as it comes off the catalyst bed. The quenching inhibits further undesired side reactions due to the high temperatures, by rapidly cooling the process stream.

During the process of dehydrogenation of paraffins, the catalyst accrues a coke buildup over time. The coke buildup eventually adversely affects the catalyst performance and the catalyst needs to be regenerated. The catalyst is cycled through a continuous catalyst regenerator as part of the system for the paraffin dehydrogenation. Simple air-burn regeneration returns fresh catalyst performance. The regeneration can take place at ambient pressure using air, or can be at higher pressures using air, or another oxidation agent, such as oxygen, although air is preferred.

Some dehydrogenation processes use a noble metal catalyst, while others use a non-noble metal catalyst. Zeolites are also used. The catalyst for fluidized bed reactors comprises small particles that are typically in the range of approximately 75 micrometers.

Another choice for the catalyst is a metal oxide stabilized zirconia. The metal in the metal oxide for stabilization can include metals such as scandium, yttrium, lanthanum, cerium, actinium, calcium, and magnesium.

We discovered that zirconia catalyst is sensitive to low levels of water in the process stream of a dehydrogenation process. For example, several hundred ppm of water significantly reduced the zirconia catalyst activity. When the catalyst is regenerated, coke is burned off the catalyst and additional fuel for reheat is burned over the fluidized catalyst. The amount of water contained in the combustion products will largely deactivate the catalyst under dehydrogenation reaction conditions.

The effect on the catalyst can be reversed by drying the catalyst in propane or hydrogen for about 10 to about 30 min which restores the catalyst to full activity. However, dryers in commercial operations would be very large, and would likely render the process non-competitive economically.

The invention provides an economical way to reduce or avoid water-induced activity loss of a catalyst that is being returned to a reactor from an upstream catalyst regeneration step. It can be applied to a variety of processes and dehydrogenation catalysts where water or other components need to be prevented from adsorbing over the catalyst surface or need to be removed from the catalyst surface to ensure adequate catalyst performance.

The present invention takes advantage of the adsorption equilibrium of water over a zirconia surface. Experimentation indicated that water is strongly adsorbed at typical dehydrogenation reactor conditions, e.g., about 580-620° C., but that little water remains adsorbed above about 700° C.

Experimentation also demonstrated that both total conversion and total selectivity dropped significantly when more than 1000 ppm of water was present in a propane feed to a dehydrogenation reactor using a zirconia catalyst at a temperature of 620° C. Lesser drops in conversion were seen at about 800 ppm, with the drop in conversion continuing to decrease as the water level was reduced.

Furthermore, as the temperature increases above 620° C., the reduction in conversion decreases, and it is eliminated by 700° C., even at high LHSV (e.g., 30 hr⁻¹).

Based on this experimentation, it was determined that the adsorbed water could be removed from the catalyst by heating to a temperature sufficient to remove the adsorbed water, followed by a simple purge (without a drying step) to remove the water vapor surrounding the catalyst particles if the purge is performed at or above the temperature at which very little or essentially no water remains adsorbed over the catalyst surface. In a drying step, the water which is physisorbed or chemisorbed on the catalyst surface is desorbed. The desorption kinetics determine the minimum time required for drying. In contrast, in a gas purge step, the majority of the water is assumed to be in the vapor phase, and it is simply pushed out. It is not the primary objective to desorp water from the catalyst surface; the volume between the catalyst particles is replaced. Thus, the zirconia water adsorption equilibrium determines the minimum regenerator operating temperature.

The regenerator can be operated such that the catalyst exits the regenerator at a minimum temperature of at least about 650° C., or at least about 660° C., or at least about 670° C., or at least about 680° C., or at least about 690° C., or at least about 700° C., or at least about 710° C., or at least about 720° C. Although the catalyst is only partially active if combustions products are purged at 650° C., this may be acceptable in some circumstances. Higher temperatures remove more of the adsorbed water and, thus, are more desirable.

The temperature of the catalyst exiting the regenerator can be controlled by controlling the amount of fuel burned in the regenerator and/or the catalyst circulation rate.

The regenerated catalyst is then separated from the flue gas. The regenerated catalyst is maintained at the regeneration temperature. By “maintained at the regeneration temperature,” we mean that the temperature of the regenerated catalyst does not drop below the minimum temperature sufficient to remove adsorbed water from the regenerated catalyst, even if the temperature of the catalyst does drop somewhat. There may be some unintended heat loss during the separation and transfer to the purge zone.

Regeneration of the catalyst is followed by a standard dry gas purge. The gas purge takes place at a temperature sufficient to remove the water vapor surrounding the regenerated catalyst. Desirably, the gas purge takes place at a temperature of at least about 650° C., or at least about 660° C., or at least about 670° C., or at least about 680° C., or at least about 690° C., or at least about 700° C., or at least about 710° C., or at least about 720° C. A temperature sufficient to remove adsorbed water would also be sufficient to remove the water vapor surrounding the regenerated catalyst. However, a temperature lower than the regeneration temperature would also be sufficient to remove the water vapor surrounding the regenerated catalyst. Therefore, the purge temperature is typically the same as, or less than, the regeneration temperature. The purge temperature could be higher than the regeneration temperature; however, that would unnecessarily increase operating costs.

After the dry gas purge, the purged catalyst stream entering the dehydrogenation reactor desirably has less than about 500 ppm water, or less than about 250 ppm, or less than about 200 ppm, or less than about 150 ppm, or less than about 100 ppm, or less than about 90 ppm, or less than about 80 ppm, or less than about 70 ppm, or less than about 60 ppm, or less than about 50 ppm, or less than about 40 ppm, or less than about 30 ppm, or less than about 20 ppm, or less than about 10 ppm.

The purged catalyst is maintained at the purge temperature until it is introduced into the dehydrogenation reactor. By “maintained at the purge temperature,” we mean that the temperature of the purged catalyst does not drop below the minimum temperature sufficient to remove water vapor from the regenerated catalyst, even if the temperature of the catalyst does drop somewhat. There may be some unintended heat loss during the gas purge and transfer from the regenerator to the dehydrogenation reactor, although desirably the catalyst is not cooled during the gas purge. The purged catalyst should enter the dehydrogenation reactor at a temperature of at least about 650° C., or at least about 660° C., or at least about 670° C., or at least about 680° C., or at least about 690° C., or at least about 700° C., or at least about 710° C., or at least about 720° C.

For example, in some embodiments, the regeneration temperature is at least about 720° C., and the purge temperature is at least about 700° C. As indicated about other combinations of regeneration temperature and purge temperature can be used.

The dry gas purge will typically have a flow rate of at least about one void volume displacement, or at least about twice the void volume displacement, or more. The dry gas can be any suitable dry inert gas such as nitrogen, hydrogen, CO₂, methane, inert gas, or combinations thereof.

Although the method is described with reference to the method and apparatus described in U.S application Ser. Nos. 13/681,914, 13/681,945, and 12/926,969, it should be understood that the method is not limited to either that method or apparatus. It can be use with any method or apparatus using a dehydrogenation catalyst where water could adsorb onto the catalyst and adversely affect the reaction.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method of regenerating spent dehydrogenation catalyst comprising:
heating the spent dehydrogenation catalyst in a regeneration zone to a regeneration temperature sufficient to remove coke and adsorbed water from the spent dehydrogenation catalyst to regenerate the spent dehydrogenation catalyst;
separating the regenerated catalyst from flue gas while maintaining the regenerated catalyst at the regeneration temperature; and
exposing the regenerated catalyst to a dry purge gas at a purge temperature sufficient to remove water vapor surrounding the regenerated catalyst, the purged catalyst being maintained at the purge temperature.

2. The method of claim 1 further comprising determining the regenerated catalyst temperature and the purge temperature from a dehydrogenation catalyst/water adsorption equilibrium.

3. The method of claim 1 further comprising controlling a flow rate of fuel to the regeneration zone, or a flow rate of catalyst to the regeneration zone, or both to maintain the regenerated catalyst at the regeneration temperature.

4. The method of claim 1 wherein a flow rate of the dry purge gas is at least about one void volume displacement.

5. The method of claim 1 wherein the dry purge gas is nitrogen, hydrogen, CO₂, methane, inert gas, or combinations thereof.

6. The method of claim 1 wherein the dehydrogenation catalyst is zirconia.

7. The method of claim 6 wherein the regeneration temperature is at least about 650° C., and the purge temperature is at least about 650° C.

8. The method of claim 6 wherein the regeneration temperature is at least about 700° C., and the purge temperature is at least about 700° C.

9. The method of claim 1 further comprising introducing the purged catalyst into a dehydrogenation reaction zone.

10. The method of claim 1 wherein a purged catalyst stream has less than about 200 ppm water in the vapor phase.

11. A method of dehydrogenating a hydrocarbon stream comprising:
contacting the hydrocarbon stream with a zirconia catalyst in a dehydrogenation reaction zone under dehydrogenation conditions forming a reaction product comprising olefins and spent zirconia catalyst;
removing the reaction product from the dehydrogenation reaction zone;
removing the spent zirconia catalyst from the dehydrogenation reaction zone;
heating the spent zirconia catalyst in a regeneration zone to a regeneration temperature sufficient to remove coke and adsorbed water from the spent zirconia catalyst to regenerate the spent zirconia catalyst;
separating the regenerated catalyst from flue gas while maintaining the regenerated catalyst at the regeneration temperature;
exposing the regenerated catalyst to a dry purge gas at a purge temperature sufficient to remove water vapor surrounding the regenerated catalyst, the purged catalyst being maintained at the purge temperature; and
introducing the purged catalyst into the dehydrogenation reaction zone at the purge temperature.

12. The method of claim 11 further comprising recovering the reaction product.

13. The method of claim 11 further comprising determining the regenerated catalyst temperature and the purge temperature from a zirconia/water adsorption equilibrium.

14. The method of claim 11 wherein the regeneration temperature is at least about 650° C., and the purge temperature is at least about 650° C.

15. The method of claim 11 wherein the regeneration temperature is at least about 700° C., and the purge temperature is at least about 700° C.

16. The method of claim 11 wherein a purged catalyst stream has less than about 200 ppm water in the vapor phase.

17. The method of claim 11 further comprising controlling a flow rate of fuel to the regeneration zone, or a flow rate of catalyst to the regeneration zone, or both to maintain the regenerated catalyst at the regeneration temperature.

18. The method of claim 11 wherein a flow rate of the dry purge gas is at least about one void volume displacement.

19. The method of claim 11 wherein the dry purge gas is nitrogen, hydrogen, CO₂, methane, inert gas, or combinations thereof.

20. The method of claim 11 wherein the dehydrogenation reaction zone is operated countercurrently.
